(19) United States
(12) Patent Application Publication
(10) Pub. No.: US 2015/0283237 A1
(43) Pub. Date: Oct. 8, 2015

(54) CTLA-4 BLOCKADE WITH METRONOMIC CHEMOTHERAPY FOR THE TREATMENT OF CANCER

(71) Applicants: Mitchell S. Felder, Hermitage, PA (US); Giulio Francia, El Paso, TX (US); Robert Arthur Kirken, El Paso, TX (US)

(72) Inventors: Mitchell S. Felder, Hermitage, PA (US); Giulio Francia, El Paso, TX (US); Robert Arthur Kirken, El Paso, TX (US)

(21) Appl. No.: 14/676,855

(22) Filed: Apr. 2, 2015

Related U.S. Application Data
(60) Provisional application No. 61/973,908, filed on Apr. 2, 2014.

(51) Int. Cl. A61K 39/395; A61K 31/7068; A61K 31/675

(52) U.S. Cl. CPC A61K 39/3955; A61K 31/675; A61K 31/7068; A61K 2039/505

(57) ABSTRACT

The present invention is directed to methods for treating cancer by administering an anti-CTLA-4 agent in combination with a metronomic chemotherapy or a sequential chemotherapy.

This application claims priority to U.S. Provisional Patent Application Ser. No. 61/973,908 filed April 2, 2014, which is incorporated herein by reference in its entirety.

### BACKGROUND

In 2010, 14 years after the first report of CTLA-4 blockade causing a tumor response in preclinical models (Leach et al. Science, 1996, 271, 1734-6), the United States Food and Drug Administration approved the anti-CTLA-4 antibody “ipilimumab” for the treatment of non-resectable or metastatic melanoma (Hodi et al. N Engl J Med., 2010, 363, 711-23; Pardoll, Nat Rev Cancer 2012, 12, 252-64). This approval was a pivotal moment for cancer immunotherapy (Li et al. Exp Hematol Oncol., 2013, 2, 33; Pardoll, Nat Rev Cancer 2012, 12, 252-64), a field since enriched by additional targets for therapy, such as PD-1 and LAG-3 (Li et al. Exp Hematol Oncol., 2013, 2, 33; Pardoll, Nat Rev Cancer 2012, 12, 252-64).

However, despite these successes, there are still hurdles to be overcome in the quest for optimal anti-CTLA-4 regimens, including how to minimize the likelihood of the development of autoimmune toxicity (Li et al. Exp Hematol Oncol., 2013, 2, 33; Maker et al. J Immunother, 2006, 29, 455-63).

### SUMMARY

Certain embodiments are directed to methods for treating cancer comprising administering an effective amount of an anti-CTLA-4 agent. In certain aspects the cancer specifically excludes melanoma. In a further aspect the cancer is breast cancer. In certain embodiments the anti-CTLA-4 agent is antibody ipilimumab.

Certain embodiments are directed to methods for treating breast cancer comprising administering an effective amount of metronomic chemotherapy combined with an effective amount of anti-CTLA-4 therapy. In certain respects the metronomic chemotherapy specifically excludes an upfront bolus dose—a metronomic chemotherapy with no upfront bolus dose. In certain further embodiments the metronomic chemotherapy agent is cyclophosphamide (CTX). In certain other embodiments the anti-CTLA-4 agent is antibody ipilimumab. Metronomic chemotherapy is a treatment regime where chemotherapeutic agents are administered long-term at relatively low doses, and with no or limited drug-free breaks. The doses are low enough that side effects are minimized. Metronomic chemotherapy is distinct from the maximum tolerated dose (MTD) method typically used, because traditional chemotherapy regimens call for higher doses often limited largely by the body's capacity to handle the side effects, and for limited campaigns of several weeks in order to avoid drug resistance and avoid harming the body's organs beyond a certain limit.

In certain aspects the methods comprise treating breast cancer with a regimen of CTLA-4 blockade followed by chemotherapy. In a further aspect CTLA-4 blockade is provided by antibody ipilimumab.

Certain embodiments are directed to methods of treating breast cancer comprising administering an effective anti-CTLA-4 agent in combination with metronomic chemotherapy. In certain aspects anti-CTLA-4 agent is administered at a dose of between 50, 100, 150, 200, 250, 300 to 250, 300, 350, 400, 450, 500, 550, 600 mg/day, including all values and ranges there between. In certain aspects at most or about 50, 100, 150, 200, 250, 300, 350, 400, 450, 500, 550, or 600 mg of anti-CTLA-4 agent is administered. In a further aspect the dose of anti-CTLA-4 agent is administered in one administration or in multiple administrations over 0.5, 1, 2, 3, 4, 5, 6, 7, 8, 9, or 10 minutes, hours or days. In certain aspects metronomic chemotherapy is administered at a dose of between 1, 5, 10, 20, 30, to 20, 30, 40, 50 mg/kg/day, including all values and ranges there between. In certain aspects 10, 50, 100, 150, 200, 250, 300, 350, 400, 450, 500, 550, or 600 mg of metronomic chemotherapy is administered every 1, 2, 3, 4, 5, 6, 7, or more days or weeks. In a further aspect there will be at least 1, 2, 3, 4, 5, 6, 7, 8, 9, 10, days between doses. In certain aspects the chemotherapy is administered periodically for at least 20, 30, 40, 50, 60, 70, 80, 90, 100 days or more. In a further aspect the dose of metronomic chemotherapy is administered in one dose or in multiple doses over 0.5, 1, 2, 3, 4, 5, 6, 7, 8, 9, or 10 minutes, hours, days, weeks, or months.

Individual administration refers to the compounds being formulated is separate formulations. The compounds, when administered individually, can be administered at the same time or within about 1, 2, 3, 4, 5, 6, 7, 8, 9, or 10 minutes, hours, or days. In certain aspects anti-CTLA-4 agent and metronomic chemotherapy are formulated in the same composition. The anti-CTLA-4 agent, metronomic chemotherapy, or anti-CTLA-4 agent and metronomic chemotherapy can be formulated as a tablet, a capsule, a concentrate, a powder, or a solution. In certain aspects CTLA-4 agent, metronomic chemotherapy, or CTLA-4 and metronomic chemotherapy are administered intravascularly. In a further aspect the CTLA-4 agent is antibody ipilimumab. In still a further aspect the metronomic chemotherapy is metronomic cyclophosphamide (CTX) chemotherapy.

In certain aspects administration of anti-CTLA-4 agent is prior to administration of metronomic chemotherapy. In a further aspect administration of anti-CTLA-4 agent is concurrent with metronomic chemotherapy. In still a further aspects administration of metronomic chemotherapy is prior to administration of anti-CTLA-4 agent. In a further aspect the CTLA-4 agent is antibody ipilimumab. In still a further aspect the metronomic chemotherapy is metronomic cyclophosphamide (CTX) chemotherapy.

Certain embodiments are directed to methods for treating breast cancer in a patient in need thereof comprising (i) administering a CTLA-4 blockade agent and (ii) after administration of the CTLA-4 blockade agent administering gemcitabine chemotherapy. Gemcitabine can be administered at least 1, 2, 3, 4 times in every 1, 2, 3 4, 5, 6, 7, 8, 9, 10 weeks or more. Gemcitabine can be administered at a dose of 50 mg/kg/day to 300 mg/kg/day.

A “therapeutically effective amount” in reference to the treatment of cancer, means an amount capable of invoking one or more of the following effects: (1) inhibition, to some extent, of cancer or tumor growth, including slowing down growth or complete growth arrest; (2) reduction in the number of cancer or tumor cells; (3) reduction in tumor size; (4) inhibition (i.e., reduction, slowing down, or complete stopping) of cancer or tumor cell infiltration into peripheral organs; (5) inhibition (i.e., reduction, slowing down, or complete stopping) of metastasis; (6) enhancement of anti-tumor immune response, which may, but is not required to, result in the regression or rejection of the tumor, or (7) relief, to some extent, of one or more symptoms associated with the cancer or tumor. The therapeutically effective amount may vary according to factors such as the disease state, age, sex and weight of the individual and the ability of one or more anti- cancer agents to elicit a desired response in the individual. A “therapeutically effective amount” is also one in which any toxic or detrimental effects are outweighed by the therapeutically beneficial effects.

The phrases “treating cancer” and “treatment of cancer” mean to decrease, reduce, or inhibit the replication of cancer cells; decrease, reduce or inhibit the spread (formation of metastases) of cancer; decrease tumor size; decrease the number of tumors (i.e. reduce tumor burden); lessen or reduce the number of cancerous cells in the body; prevent recurrence of cancer after surgical removal or other anti-cancer therapies; or ameliorate or alleviate the symptoms of the disease caused by the cancer.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Impact of bolus plus low-dose cyclophosphamide (CTX) combined with anti-CTLA-4 therapy on the growth of subcutaneously implanted EMT-6/P tumors. (A) Murine EMT-6/P cells were implanted s.c. in female BALB/c mice. Therapies began when tumors reached 50 mm3; the mice received control saline (i.p.), anti-CTLA-4, bolus plus low-dose CTX, or the combination of anti-CTLA-4 together with bolus plus low-dose CTX. (B) Mouse weights, as a measure of toxicity of the different treatments on the hosts. (C) Impact of the different therapies, as assessed by analysis of event-free survival (Kaplan-Meier analysis), where duration of event-free survival was defined as time to primary tumor progression beyond 1,200 mm³ or >15% weight loss. Significant event-free survival was observed with anti-CTLA-4 therapy, but this benefit was reduced by the addition of bolus +low dose CTX. The sole survivor, by day 46, in the anti-CTLA-4 therapy group was still alive and tumor-free at day 200 after tumor cell injection.

FIG. 2. Effective combination of chemotherapy with anti-CTLA-4 therapy for the inhibition of the growth of subcutaneously implanted EMT-6/P tumors. (A) Murine EMT- 6/P cells were implanted s.c. in female BALB/c mice. Therapies began when tumors reached 50 mm³; the mice received control saline (i.p.), anti-CTLA-4, bolus plus low-dose CTX, metronomic CTX, or the combination of anti-CTLA-4 together with metronomic CTX. One additional group received anti-CTLA-4 therapy as a first-line treatment and then, when the tumors began to relapse at around day 21, a second-line therapy consisting of gemcitabine (160 mg/kg every 3 days, i.p.), starting on day 21. (B) Mouse weights, as a measure of toxicity of the different treatments on the hosts. (C) Impacts of the different therapies, as assessed by analysis of event-free survival (Kaplan-Meier analysis), where duration of event-free survival was defined as time to primary tumor progression beyond 1,200 mm³ or >15% weight loss. Significant event-free survival was observed with anti-CTLA-4 therapy, and this benefit was improved by the combination of anti-CTLA-4 therapy plus metronomic CTX, or by the sequential regimen, using first-line of anti-CTLA-4 followed by gemcitabine chemotherapy in the relapsing tumors.

### DESCRIPTION

Ipilimumab is used for the treatment of non-resectable metastatic melanoma, and clinical trials are ongoing to test its use for the treatment of other malignancies (according to clinicaltrials.gov), including lung cancer, prostate cancer (Kwek et al., Nat Rev Cancer 2012, 12:289-97), and breast cancer. Furthermore, several clinical trials are actively recruiting, or are ongoing, to evaluate different combinations of chemotherapy and ipilimumab in melanomas and other cancer types.

Extensive experimental therapeutic analysis of metronomic chemotherapy (Bocci et al., Neoplasia, 2012, 14:833-45; Chow et al., Invest New Drugs, 2014, 32:47-59; Hackl et al., Gut, 2013, 62:259-71; Francia et al., Mol Cancer Ther., 2012, 11:680-9; Francia et al., Clin Cancer Res., 2009, 15:6358-66, Emmenegger et al., Neoplasia, 2011, 13:40-8; Emmenegger et al., Mol Cancer Ther., 2007, 6:2280-89; Tang et al., Neoplasia, 2010, 12:928-40), including the use of CTX chemotherapy (Kerbel and Kamen, Nat Rev Cancer, 2004, 4:423-36) with an upfront bolus CTX dose (Shaked et al., Cancer Res., 2005, 65:7045-51), and the use of sequential chemotherapy regimens, as well as second-line therapies (du Manoir et al., Clin Cancer Res., 2006, 12:904-16) has been reported.

Regarding metronomic chemotherapy, its proposed mechanisms of action are many (Bocci et al., Proc Natl Acad Sci USA, 2003, 100:12917-22; Kerbel and Kamen, Nat Rev Cancer, 2004, 4:423-36; Pasquier et al., Nat Rev Clin Oncol., 2010, 7:455-65; Francia et al., Mol Cancer Ther., 2012, 11:680-89), including inhibition of angiogenesis and inhibition of cancer stem cell growth, and activation of the immune system (Chen et al., Mol Ther., 2010, 18:1233-43; Ghiringhelli et al., Cancer Immunol Immunother., 2007, 56:641-48; Kerbel and Kamen, Nat Rev Cancer, 2004, 4:423-36). Regarding the latter, this has been documented for the drugs cyclophosphamide and gemcitabine (Lesterhuis et al., PLoS One, 2013, 8:e61895), although it remains to be determined the extent to which metronomic dosing of other clinically used chemotherapy drugs may activate the immune system.

Because ipilimumab therapy is directed at immune activation, and because combinations of ipilimumab with chemotherapy agents are being evaluated clinically, whether the immune activation activity of CTLA-4 blockade could be augmented by the addition of metronomic chemotherapy was investigated. For example, treatment with CTLA-4 might be followed by a metronomic chemotherapy maintenance treatment, which would be an interesting concept, given the relatively low toxicity profile of metronomic chemotherapy regimens (Kerbel and Kamen, Nat Rev Cancer, 2004, 4:423-36; Pasquier et al., Nat Rev Clin Oncol., 2010, 7:455-65).

An effective metronomic-type regimen combination was investigated. A protocol was developed in 2005 (Shaked et al., Cancer Res, 2005, 65:7045-51), involving an upfront bolus CTX dose, immediately followed by a metronomic CTX regimen, by placing CTX in the mice's drinking water (Shaked et al., Cancer Res, 2005, 65:7045-51; Man et al. Cancer Res., 2002, 62:2731-35).

This bolus in combination with low dose CTX approach actually caused a less effective tumor response than anti-CTLA-4 monotherapy. These results thus serve as a cautionary note against the use of a bolus plus metronomic cyclophosphamide component in therapies involving CTLA-4 blockade.

The bolus plus metronomic chemotherapy could be improved by the addition of a targeting agent such as the anti-VEGFR2 antibody DC 101 (Francia et al., Mol Cancer Ther., 2008, 7:3452-59). It was believed that a bolus plus metronomic regimen hinders the efficacy of a targeted therapy.

Two alternative strategies were tested. One was to omit the bolus upfront CTX dose, and administer a metronomic low-dose CTX protocol. The second was to adopt gemcitabine chemotherapy, with which notable responses in a preclinical breast cancer model was observed (Francia et al., Mol Cancer Ther., 2012, 11:680-89) but to separate its administration from that of the CTLA-4 antibody.

Thus in certain embodiments treatment with CTLA-4 antibody was combined with sequential gemcitabine therapy. In certain aspects the CTLA-4 antibody was co-administered with metronomic CTX. The results suggest that both strategies can improve a CTLA-4 monotherapy regimen, with the sequential gemcitabine therapy generating more potent anti-tumor responses in a model.

It is not unprecedented for two conceptually different anti-cancer strategies to produce seemingly counterintuitive results when they are combined, as observed in this study. For example, prior to the study of Teicher et al. (Cancer Res., 1992, 52:6702-04), an argument against the combination of conventional cytotoxic chemotherapy drugs with anti-angiogenic agents was that the latter would reduce vascular permeability into tumors and therefore hinder the delivery of chemotherapy into a tumor mass. However, to the contrary, when Teicher et al. (Cancer Res., 1992, 52:6702-04) evaluated such a combination therapy, they observed that anti-angiogenic drugs could actually enhance the antitumor effect of chemotherapy.

Regarding the results presented herein, it was expected that bolus plus metronomic CTX would increase the efficacy of anti-CTLA-4 therapy—but the results proved otherwise. This was unexpected, because it was previously reported that bolus with low dose CTX could enhance the antitumor effect of oral gemcitabine prodrug (Francia et al., Mol Cancer Ther., 2012, 11:680-89). Nonetheless, it was observed that some chemotherapy regimens can be effectively combined with anti-CTLA-4 therapy. The results are consistent with other studies, such as that by Mokyr et al. (Cancer Res., 1998, 58:5301-04), which showed that low-dose melphalan could be combined effectively with anti-CTLA-4 therapy.

Interestingly, Lesterhuis et al. (PLoS One, 2013, 8:e61895) reported that CTLA-4 can be administered concurrently with gemcitabine to produce significant anti-tumor responses. Similarly, Jure-Kunkel et al. (Cancer Immunol Immunother., 2013, 62:1533-45) showed an effective combination of anti-CTLA-4 plus chemotherapy (in which their standard protocol was to administer the anti-CTLA-4 antibody 1 day after the administration of chemotherapy), including gemcitabine. Such a regimen was considered, because of the bolus with low dose CTX results. Overall, the results show that chemotherapy can augment the impact of anti-CTLA-4 therapy, but that caution is necessary in the design of such combinations, because some may be counterproductive.

### I. TREATMENT OF CANCER

The inventors have shown that metronomic chemotherapy, with no upfront bolus dose, combined with anti-CTLA-4 therapy can be used to treat cancer. In certain aspects the cancer is a bladder, blood, bone, bone marrow, brain, breast, colorectal, esophagus, gastrointestine, head, kidney, liver, lung, nasopharynx, neck, ovary, pancreas, prostate, skin, stomach, testicular, tongue, or uterine cancer. In still a further aspect the cancer is breast cancer.

In certain embodiments, the invention also provides compositions comprising one or more anti-cancer agents in a pharmaceutically acceptable formulation. Thus, the use of one or more anti-cancer agents that are provided herein in the preparation of a medicament is also included. Such compositions can be used in the treatment of a variety of cancers. In certain embodiments the treatment is for a metastatic cancer, e.g., lung, breast, or prostate cancer.

The anti-cancer agents may be formulated into therapeutic compositions in a variety of dosage forms such as, but not limited to, liquid solutions or suspensions, tablets, pills, powders, and injectable or infusible solutions. The preferred form depends upon the mode of administration and the particular disease targeted. The compositions also preferably include pharmaceutically acceptable vehicles, carriers, or adjuvants, well known in the art.

Acceptable formulation components for pharmaceutical preparations are nontoxic to recipients at the dosages and concentrations employed. In addition to the anti-cancer agents that are provided, compositions may contain components for modifying, maintaining, or preserving, for example, the pH, osmolarity, viscosity, clarity, color, isotonicity, odor, sterility, stability, rate of dissolution or release, adsorption, or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (see Remington's Pharmaceutical Sciences, 18 th Ed., (A. R. Gennaro, ed.), 1990, Mack Publishing Company), hereby incorporated by reference.

Formulation components are present in concentrations that are acceptable to the site of administration. Buffers are advantageously used to maintain the composition at physiological pH or at a slightly lower pH, typically within a pH range of from about 4.0 to about 8.5, or alternatively, between about 5.0 to 8.0. Pharmaceutical compositions can comprise TRIS buffer of about pH 6.5-8.5, or acetate buffer of about pH 4.0-5.5, which may further include sorbitol or a suitable substitute therefor.

The pharmaceutical composition to be used for in vivo administration is typically sterile. Sterilization may be accomplished by filtration through sterile filtration membranes. If the composition is lyophilized, sterilization may be conducted either prior to or following lyophilization and reconstitution. The composition for parenteral administration may be stored in lyophilized form or in a solution. In certain embodiments, parenteral compositions are placed into a container having a sterile access port, for example, an intravenous solution bag or vial having a stopper pierceable by a hypodermic injection needle, or a sterile pre-filled syringe ready to use for injection.

The above compositions can be administered using conventional modes of delivery including, but not limited to, intravenous, intraperitoneal, oral, intralymphatic, subcutaneous administration, intraarterial, intramuscular, intrapleural, intrathecal, and by perfusion through a regional catheter. Local administration to a tumor or a metastasis in question is also contemplated by the present invention. When administering the compositions by injection, the administration may be by continuous infusion. For parenteral administration, the agents may be administered in a pyrogen-free, parenterally acceptable aqueous solution comprising the desired anti-cancer agents in a pharmaceutically acceptable vehicle. A particularly suitable vehicle for parenteral injection is sterile distilled water in which one or more anti-cancer agents are formulated as a sterile, isotonic solution, properly preserved.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

If desired, stabilizers that are conventionally employed in pharmaceutical compositions, such as sucrose, trehalose, or glycine, may be used. Typically, such stabilizers will be added in minor amounts ranging from, for example, about 0.1% to about 0.5% (w/v). Surfactant stabilizers, such as TWEEN®-20 or TWEEN®-80 (ICI Americas, Inc., Bridgewater, N.J., USA), may also be added in conventional amounts.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents. Compositions for parental administration are also sterile, substantially isotonic and made under GMP conditions.

Therapeutically effective doses will be easily determined by one of skill in the art and will depend on the severity and course of the disease, the patient's health and response to treatment, the patient's age, weight, height, sex, previous medical history and the judgment of the treating physician.

In some methods of the invention, the cancer cell is a tumor cell. The cancer cell may be in a patient. The patient may have a solid tumor. In such cases, embodiments may further involve performing surgery on the patient, such as by resecting all or part of the tumor. Compositions may be administered to the patient before, after, or at the same time as surgery. In additional embodiments, patients may also be administered directly, endoscopically, intratracheally, intratumorally, intravenously, intralesionally, intramuscularly, intraperitoneally, regionally, percutaneously, topically, intrarterially, intravesically, or subcutaneously. Therapeutic compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months.

Dosing. The amount of active compound administered may be dependent on the subject being treated, the subject's weight, the manner of administration and the judgment of the prescribing physician. For example, a dosing schedule may involve the daily or semi-daily administration of the compound at a perceived dosage of about 1 μg to about 1000 mg. In another embodiment, intermittent administration, such as on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. The dosages, however, may be varied depending upon the requirements of the patient, the severity of the condition being treated, and the compound being used.

In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al. Cancer Chemother Reports, 1966, 50:219-44) and the following Table for Equivalent Surface Area Dosage Factors. See also Reagan-Shaw et al. FASEB J., 2007, 22:659-61, regarding dose translation from animal to human studies).

TABLE 1
Approximate Equivalent Surface Area Dosage Factors

| | To | | | | |
|---|---|---|---|---|---|
| | Mouse | Rat | Monkey | Dog | Human |
| From | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | ⅗ | 1 | ½ |
| Human | 12 | 7 | 3 | 2 | 1 |

The dosage of such compounds lies preferably within a range of circulating concentrations that include the ED5o with little or no toxicity. The dosage may vary within this range depending upon the dosage form employed and the route of administration utilized.

Generally, a therapeutically effective amount may vary with the subject's age, condition, and gender, as well as the severity of the medical condition in the subject. The dosage may be determined by a physician and adjusted, as necessary, to suit observed effects of the treatment.

Methods of treating cancer may further include administering to the patient chemotherapy or radiotherapy, which may be administered more than one time. Chemotherapy includes, but is not limited to, cisplatin (CDDP), carboplatin, procarbazine, mechlorethamine, cyclophosphamide (CTX), camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxotere, taxol, transplatinum, 5-fluorouracil, vincristin, vinblastin, methotrexate, gemcitabine, oxaliplatin, irinotecan, topotecan, or any analog or derivative variant thereof. Radiation therapy includes, but is not limited to, X-ray irradiation, UV-irradiation, γ-irradiation, electron-beam radiation, or microwaves. Moreover, a cell or a patient may be administered a microtubule stabilizing agent, including, but not limited to, taxane, as part of methods of the invention. It is specifically contemplated that any of the compounds or derivatives or analogs, can be used with these combination therapies.

### II. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE I

### Anti-ctla-4 Therapy Combined with Low-Dose CTX

The combination of CTLA-4 blockade with metronomic chemotherapy regimens was evaluated. Murine EMT-6 breast tumor cells were subcutaneously implanted into syngeneic BALB/c mice (n=6-8/group) and therapies on the established tumors were evaluated. Murine CTLA-4 blockade was achieved using anti-mouse CD152 (CTLA-4), clone 9H10, injected on day 1 (100 μg/mouse) and on day 6 (35 μg/mouse) of therapy. Anti-CTLA-4 therapy was administered alone or combined with metronomic regimens. These included: (a) bolus (150 mg/kg, i.p.) CTX followed by metronomic CTX (20 mg/kg/day, p.o.), (b) metronomic CTX, and (c) sequential gemcitabine therapy (160 mg/kg every 3 days, i.p.) given when the tumors relapsed after the anti-CTLA-4 therapy.

The regimen involving first-line anti-CTLA4 therapy followed by second-line gemcitabine therapy (sequential therapy), produced sustained tumor regression that continued for over 100 days. In this group, 4/6 mice did not show tumor regrowth; one mouse showed tumor regrowth under continuous gemcitabine therapy with concomitant development of lung metastasis. Tumor cells lines were derived from the relapsing tumor and from the lung metastasis. Collectively, the data show that bolus plus metronomic CTX may compromise anti-CTLA-4 therapy. Furthermore, anti-CTLA-4 therapy can be combined effectively with metronomic CTX, or with sequential gemcitabine therapy, in a breast cancer model.

### A. Results

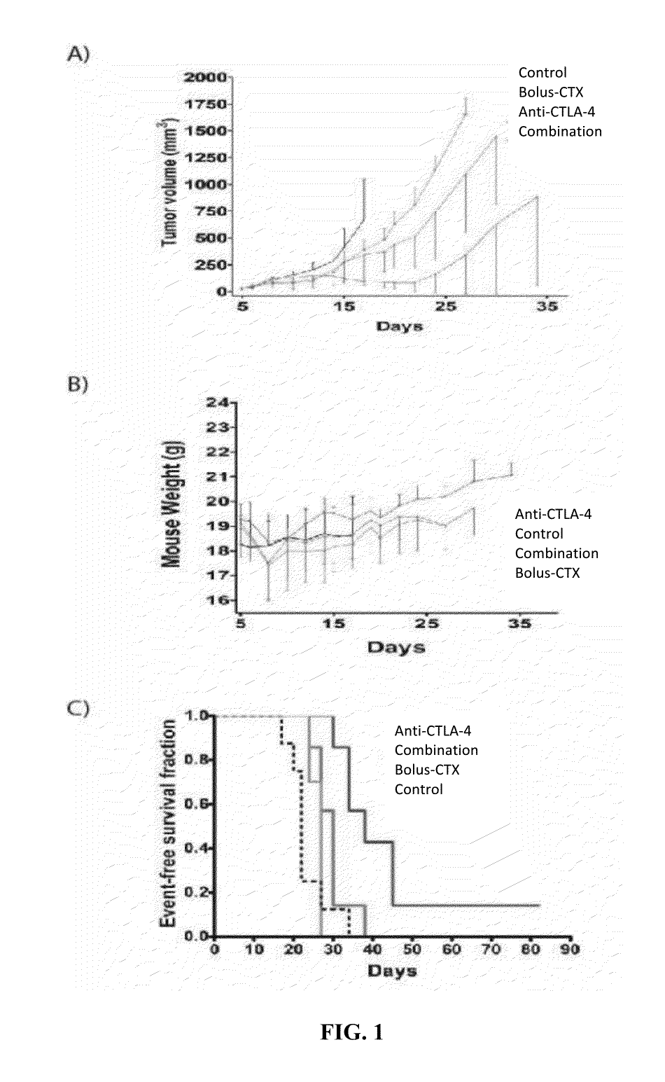

Anti-CTLA-4 therapy combined with bolus plus low-dose CTX. To evaluate whether metronomic CTX regimens could be combined effectively with an anti-CTLA-4 treatment, a combination regimen was tested on subcutaneously implanted EMT-6/P tumors (FIG. 1A). For the chemotherapy component, a bolus with low dose CTX protocol consisting of a bolus of CTX (given on day 1 as a 150 mg/kg, i.p. injection) plus low-dose CTX (20 mg/kg/day, p.o., from day 1) was used. In total, 32 mice bearing EMT-6/P tumors were divided into four groups that were treated with saline (control), bolus with low dose CTX, anti-CTLA-4 antibody, or bolus with low dose CTX plus anti-CTLA-4 antibody.

FIG. 1A shows the impact of the therapies on tumor growth. Thus, control-treated tumors grew rapidly, the bolus with low dose CTX treatment resulted in a minimal reduction in tumor growth, whereas anti-CTLA-4 antibody treatment caused tumor regression over a 20-day period, followed by tumor relapses in the subsequent 15 days.

Surprisingly, the bolus with low dose CTX plus CTLA-4 combination therapy resulted in no tumor regression and in overall tumor growth, that was only marginally slower than that observed in bolus with low dose CTX alone. Thus bolus with low dose CTX apparently interfered with the efficacy of anti-CTLA-4 therapy in this tumor model. This was an unexpected finding, because it was previously reported that bolus with low dose CTX could be combined effectively with an anti-VEGFR2 antibody (Francia et al., Mol Cancer Ther., 2008, 7:3452-59) or with a metronomic oral gemcitabine regimen (Francia et al., Mol Cancer Ther., 2012, 11:680-89). Additionally, it was noted that by day 22, the CTLA-4 antibody monotherapy resulted in complete tumor regression in two mice, one of which then began to show tumor regrowth a few days later. All anti-CTLA-4 treated tumors shrank after the therapy began, although tumor relapses were eventually observed in 5/6 mice in this group. Thus, anti-CTLA-4 therapy was effective in this tumor model, but its therapeutic efficacy was hampered by adding a bolus with low dose CTX treatment.

To assess the relative toxicity of the therapies, body weights of the mice over the course of the experiment was monitored, as in the previous studies (du Manoir et al., Clin Cancer Res., 2006, 12:904-16; Francia et al., Mol Cancer Ther., 2012, 11:680-89). FIG. 1B shows that the treatments that included a bolus with low dose CTX component produced a short-term weight loss, as reported previously (Shaked et al., Cancer Res., 2005, 65:7045-51) followed by a gain in weight in the treated mice. The tumor responses observed in this experiment was plotted as a Kaplan-Meier plot (FIG. 1C), which showed that time to 50% event-free survival with CTLA-4 antibody treatment was 38 days, compared with 22 days for the control group.

FIG. 1C also shows that one anti-CTLA-4 antibody-treated mouse that had been bearing a palpable tumor in the first 2 weeks of the experiment, showed tumor regression and remained tumor free for the rest of the follow-up period. This mouse was still alive and tumor-free 200 days later.

Anti-CTLA-4 therapy combined with low dose CTX, or with sequential gemcitabine therapy. Whether other chemotherapy regimens, either in combination with or subsequent to the anti-CTLA-4 therapy, can be incorporated was tested. The reasoning was that the bolus CTX might have been toxic to the T cell population that is activated following the CTLA-4 blockade, thus negating the therapeutic benefit of that therapy. Consequently, separating the time of the chemotherapy dosing from that of the anti-CTLA-4 antibody, or omitting the bolus chemotherapy component was considered.

A low-dose CTX, without a bolus was evaluated, as in some of the previous studies (Man et al., Cancer Res., 2002, 62:2731-5; Francia et al., Clin Cancer Res., 2009, 15:6358-66). Furthermore, since a marked preclinical tumor response in human breast cancer cells treated with a gemcitabine regimen was noted, this regimen was considered, but for it to be administered only after CTLA-4 tumors began to relapse.

To evaluate these alternative therapies, 43 mice were implanted with EMT-6/P tumors, and these were subsequently divided into seven groups. Therapies were begun when all mice had established tumors and when the average tumor volume was approximately 50 mm³.

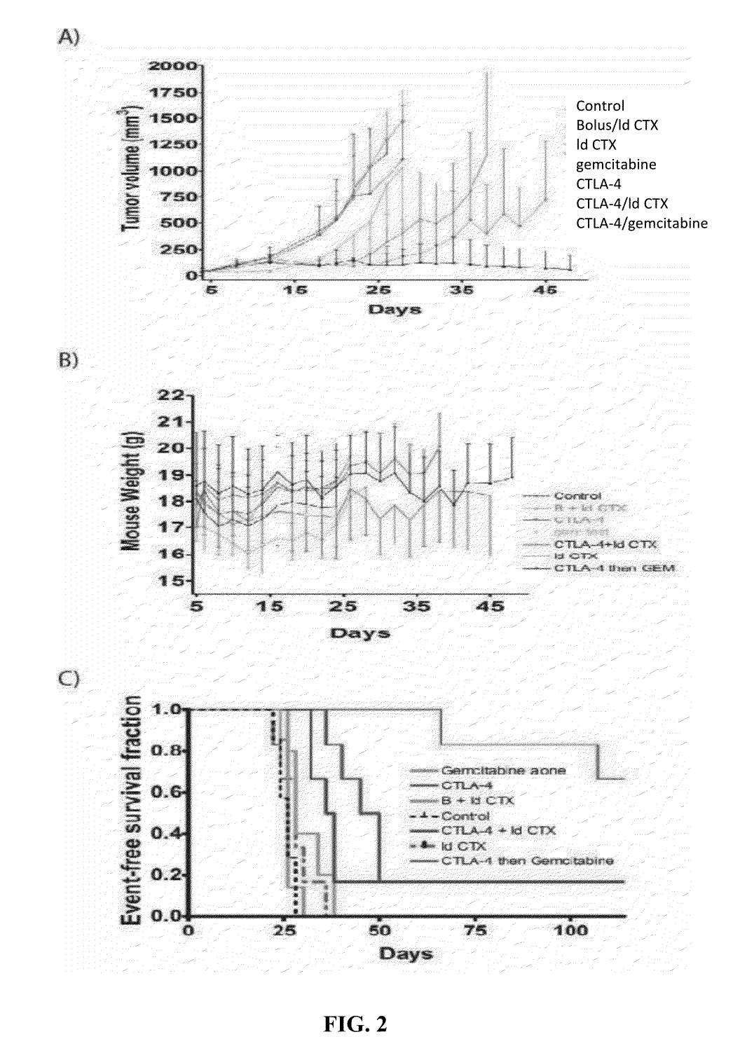

As shown in FIG. 2A, no or minimal impact of tumor growth was noted, compared with controls, in the groups treated with low-dose CTX regimens. Anti-CTLA-4 therapy led to initial tumor regression in the first 2 weeks after therapy started, followed by tumor relapse. The combination of anti-CTLA-4 plus low-dose CTX (both co-administered from day 6 onwards) produced a greater inhibition of tumor growth that was observed with the anti-CTLA-4 monotherapy.

As shown in FIG. 2A, one additional group (anti-CTLA-4 then Gem) initially received the anti-CTLA-4 antibody, which again produced tumor regression that lasted 2 weeks, followed by tumor relapse. For this group, as soon as tumors started to relapse (i.e. around day 21), the second-line therapy of gemcitabine (160 mg/kg every 3 days, i.p) was administered. On the day this second-line therapy started (i.e., day 21), all mice in this group had visible tumors and an average tumor volume of 145 mm³.

As shown in FIG. 2A, the second-line therapy caused the tumors to regress a second time, and by day 36, only 2/6 mice had palpable tumors. These two mice eventually showed tumor regrowth. The remaining four mice showed complete tumor regression and were still tumor-free 100 days later.

The relative toxicity of the tested therapies was evaluated, as show in FIG. 2B. No treatment caused significant changes in mice weights, compared with controls. The only exception was a slight weight drop seen in one group that received a single upfront dose of gemcitabine. This result initially suggested that the gemcitabine treatment might produce toxicity. However, no such toxicity was eventually observed in the CTLA-4 then gemcitabine group, after the gemcitabine treatment started. Indeed, in that group gemcitabine was continued to be administered every 3 days for a further 30 days without observing any obvious toxicity.

The results were evaluated using a Kaplan-Maier analysis (FIG. 2C), which showed the observed event-free survival in 4 of 6 mice in the CTLA-4 then gemcitabine group. Event-free survival in 1 of 6 mice for both the CTLA-4 monotherapy group and for the CTLA-4 plus ld CTX group was noted.

Regarding the CTLA-4 then gemcitabine-treated group, the two mice with visible tumors after day 36 (FIG. 3) eventually showed tumor relapses, one of these mice developed advanced lung metastases (while under gemcitabine therapy), and was sacrificed on day 107 (FIG. 3). These results show that although the anti-CTLA-4 then gemcitabine therapy was effective, tumor drug resistance eventually can develop in a subset of the mice, and that metastatic disease (here, to the lungs) can occur, under therapy, in this tumor model. The four surviving mice in this group were still alive and tumor-free 100 days later.

B. Materials and Methods

Breast Cancer Model. The EMT-6 model is generally accepted in the field of drug discovery and development research (Lesterhuis et al. 2011; Twentyman and Bleehen 1976) and has been used in the development of breast cancer drugs, such as docetaxel (Taxotere).

Drug Preparation. Gemcitabine hydrochloride was purchased from Selleck Chemicals (Houston, Tex.) and made up in sterile phosphate-buffered saline (PBS) immediately prior to i.p. administration. Cyclophosphamide was purchased from Sigma and made up in PBS prior to i.p. injection or prior to its addition to the mice's drinking water. Low-dose metronomic cyclophosphamide was administered at an estimated 20 mg/kg/day as described previously (Man et al. Cancer Res., 2002, 62:2731-35). Some regimens included an upfront bolus dose of CTX that was administered on day 1 as a 150 mg/kg i.p. injection of CTX, as described previously (Shaked et al., Cancer Res., 2005, 65:7045-51; Francia et al., Mol Cancer Ther., 2012, 11:680-89).

Antibody. Anti-mouse CD152 (CTLA-4), FG purified clone 9H10, was purchased from Ebioscience (San Diego, Calif.). See Peggs et al. JEM, 2009, 206:1717-25.

Cell lines. Murine EMT-6/P mammary carcinoma cells were grown in RPMI 1640, supplemented with 10% fetal bovine serum and 2 mM L-glutamine. Cells were maintained as monolayers in a humidified incubator at 37° C. and 5% CO2.

In vivo tumor growth assessment. Six-week-old female BALB/c mice were purchased from Harlan (Indianapolis, Ind.). Mice were allowed to acclimate for 2 weeks before implantation of tumor cells. To prepare cells for injection, subconfluent plates were harvested with 1% trypsin-EDTA (Life Technologies Bethesda Research Laboratories, Gaithersburg, Md.), and cells were then washed and resuspended in RPMI 1640 at 2×10⁶ per mL. Then, 2×10⁵ cells were injected subcutaneously, in 0.1 mL volumes, into the flanks of the mice.

Mice were monitored twice weekly for tumor growth, as measured using Vernier calipers, and calculated using the formula (length×width)/2, and for fluctuations in body weight. Institutional guidelines were followed to determine when the experimental end points were reached. Results were also plotted as event-free survival (Kaplan-Meier analysis) over time, where the duration of event-free survival was defined as time to primary tumor progression beyond 1,200 mm₃ or >15% weight loss, as in the previous study (du Manoir et al., Clin Cancer Res., 2006, 12, 904-16).

1. A method for treating cancer in a patient in need thereof comprising administering an effective amount of an anti-CTLA-4 agent in combination with metronomic chemotherapy or sequential chemotherapy.

2. The method of claim 1, wherein the anti-CTLA-4 agent is an ipilimumab.

3. The method of claim 1, wherein an upfront bolus dose of chemotherapy is specifically excluded.

4. The method of claim 1, wherein melanoma is specifically excluded.

5. The method of claim 1, wherein the cancer is breast cancer.

6. The method of claim 1, wherein the metronomic chemotherapy agent is cisplatin (CDDP), carboplatin, procarbazine, mechlorethamine, cyclophosphamide (CTX), camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxotere, taxol, transplatinum, 5-fluorouracil, vincristin, vinblastin, methotrexate, gemcitabine, oxaliplatin, irinotecan, or topotecan.

7. The method of claim 1, wherein the metronomic chemotherapy agent is cyclophosphamide (CTX).

8. The method of claim 1, wherein the anti-CTLA-4 agent is a therapeutic anti-CTLA-4 antibody and the metronomic chemotherapy agent is a cyclophosphamide (CTX).

9. A method for treating breast cancer in a patient in need thereof comprising (i) administering a CTLA-4 blockade agent and (ii) after administration of the CTLA-4 blockade agent administering gemcitabine chemotherapy.

10. The method of claim 10, wherein gemcitabine is administered at least three times in two weeks.

11. The method of claim 10, wherein gemcitabine is administered at a dose of 50 mg/kg/day to 300 mg/kg/day.

12. The method of claim 9, wherein the -CTLA-4 blockade agent is ipilimumab.

13. A method for treating breast cancer in a patient in need thereof comprising administering a CTLA-4 blockade agent and a low-dose chemotherapy for at least three weeks.

14. The method of claim 13, wherein the chemotherapy is cyclophosphamide.

15. The method of claim 13, wherein the chemotherapy is administered at a dose of 600mg or less per day.

16. The method of claim 15, wherein the chemotherapy is administered at a dose of 300mg or less per day.

17. The method of claim 13, wherein the chemotherapy is administered periodically over 30 days.

18. The method of claim 17, wherein the chemotherapy is administered periodically over 90 days.

19. The method of claim 13, wherein the anti-CTLA-4 agent is antibody ipilimumab.

20. The method of claim 13, wherein an upfront bolus dose of chemotherapy is specifically excluded.
